(19) United States
(12) Patent Application Publication
(10) Pub. No.: US 2009/0214641 A1
(43) Pub. Date: Aug. 27, 2009

(54) Sweetened Capsules for Administration

(76) Inventor: Joar Opheim, Aptos, CA (US)

Correspondence Address: JONES DAY (for Nordic Naturals), 222 EAST 41ST. STREET, NEW YORK, NY, 10017-6702, US

(21) Appl. No.: 12/037,822

(22) Filed: Feb. 26, 2008

(51) Int. Cl. A61K 9/48

(52) U.S. Cl. 424/456

(57) ABSTRACT

In an aspect of the present invention, a sweetened gelatin capsule comprises a dose and a gelatin capsule encapsulating the dose. The gelatin capsule comprises a gelatin substance, a gelatin softener, water, and a sweetener. The concentration by weight of the water in the gelatin capsule is from about 6% to about 10%. The concentration by weight of the sweetener in the gelatin capsule is from about 0.002% to about 0.003%.

### FIELD OF THE INVENTION

The present invention relates to sweetened capsules for the administration of a dose, which may be for nutritive or medicinal purposes.

### BACKGROUND OF THE INVENTION

The taste of many medicinal and nutritive components can be quite distinctive and potentially unpleasant. Improvements in the taste of certain drugs and nutritional supplements can lead to a higher rate of compliance by consumers. A higher compliance will result in greater commercial success for the drug and supplement manufacture and in increased health and well being for particular consumers.

Taste is a matter of purely subjective preference that is strongly influenced by experience and cultural impressions. Broad generalizations about consumer taste presence can thus sometimes be relied upon in predicting market acceptance of specific drug and nutritive formulations. In Norway, for example the tastes of fish oils are far more palatable than in the United States. As a consequence of this United States market aversion to the taste of fish oils, many residents of the United States may be unwilling to ingest fish oils and will therefore not benefit from the nutritional and medicinal qualities of fish oils.

Increasing the consumption of fish oils containing the recommended doses of certain omega-3 fatty acids may have a medically and nutritionally beneficial effect on many consumers and patients. The present invention addresses this need by providing sweetened capsules that can contain a fish oil dose in order to improve the palatability and therefore the consumption of the fish oil with nutritionally beneficial effects.

Discussion or citation of a reference herein will not be construed as an admission that such reference is prior art to the present application.

### SUMMARY OF INVENTION

In a first aspect, provided herein is a sweetened gelatin capsule suitable for encapsulating a dose that comprises a gelatin substance, a gelatin softener, water, and a sweetener. In certain embodiments, the concentration by weight of the water in the sweetened gelatin capsule is between 6% and 10%. In certain embodiments, the concentration by weight of the sweetener in the sweetened gelatin capsule is from about 0.002% to about 0.003%.

In some embodiments, the sweetener is a natural sweetener. In some embodiments, the natural sweetener is luo han. In some embodiments, the concentration by weight of the luo han in the sweetened gelatin capsule is about 0.00268%. In some embodiments, the concentration by weight of the sweetener in the sweetened gelatin capsule is from about 0.0024% to about 0.0028%.

In some embodiments, the sweetened gelatin capsule encapsulates a dose. In some embodiments, the dose comprises fish oil. In some embodiments, the dose comprises a sweetener, where the concentration by weight of the sweetener in the dose is between 0.002% and 0.003%. In some embodiments, the sweetener in the dose is a natural sweetener, e.g., luo han. In some embodiments, the dose comprises a flavoring. In some embodiments, the concentration by weight of the flavoring in the dose is from about 0.05% to about 5%. The flavoring can comprise a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea. In some embodiments, the flavoring is a fruit flavoring. In some embodiments, the dose comprises a medicinal component or a nutritive component.

In some embodiments, the sweetened gelatin capsule further comprises a flavoring. In some embodiments, the concentration by weight of the flavoring in the sweetened gelatin capsule is from about 0.05% to about 5%. The flavoring can comprise a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea. In some embodiments, the flavoring is a fruit flavoring.

In some embodiments, the concentration by weight of the gelatin softener in the sweetened gelatin capsule is from about 10% to about 35%. In some embodiments, the sweetened gelatin capsule further comprises a coloring agent.

In another aspect, provided herein is a sweetened gelatin capsule comprising a dose and a gelatin capsule encapsulating the dose. In certain embodiments, the gelatin capsule comprises a gelatin substance, a gelatin softener, water, and a sweetener. In certain embodiments, the concentration by weight of the water in the gelatin capsule is from about 6% to about 10%. In certain embodiments, the concentration by weight of the sweetener in the gelatin capsule is from about 0.002% to about 0.003%.

In some embodiments, the sweetener is a natural sweetener. In some embodiments, the natural sweetener is luo han. In some embodiments, the concentration by weight of the luo han in the gelatin capsule is about 0.00268%. In some embodiments, the concentration by weight of the sweetener in the gelatin capsule is from about 0.0024% to about 0.0028%.

In some embodiments, the dose comprises fish oil. In some embodiments, the dose comprises a sweetener. In some embodiments, the concentration by weight of the sweetener in the dose is between 0.002% and 0.003%. In some embodiments, the concentration by weight of the sweetener in the dose is about equal to the concentration by weight of the sweetener in the gelatin capsule. In some embodiments, the sweetener in the dose is a natural sweetener. In some embodiments, the natural sweetener in the dose is luo han.

In some embodiments, the dose comprises a flavoring. In some embodiments, the concentration by weight of the flavoring in the dose is from about 0.05% to about 5%. The flavoring can comprise a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea. In some embodiments, the flavoring is a fruit flavoring.

In some embodiments, the gelatin capsule further comprises a flavoring. In some embodiments, the concentration by weight of the flavoring in the gelatin capsule is from about 0.05% to about 5%. The flavoring can comprise a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea. In some embodiments, the flavoring is a fruit flavoring.

In some embodiments, the concentration by weight of the gelatin softener in the gelatin capsule is from about 10% to about 35%. In some embodiments, the dose comprises a medicinal component or a nutritive component. In some embodiments, the gelatin capsule further comprises a coloring agent.

In another aspect, provided herein is a kit containing a nutritional or medicinal supplement that comprises a plurality of capsules and a container for storing the plurality of capsules. In certain embodiments, each capsule in the plurality of capsules comprises a gelatin capsule encapsulating a dose. In certain embodiments, the gelatin capsule comprises a gelatin substance, a gelatin softener, water, and a sweetener. In certain embodiments, the concentration by weight of the water in the gelatin capsule is from about 6% to about 10%. In some embodiments, the concentration by weight of the gelatin softener in the gelatin capsule is from about 10% to about 35%. In certain embodiments, the concentration by weight of the sweetener in the gelatin capsule is from about 0.002% to about 0.003%.

In some embodiments, the sweetener is a natural sweetener. In some embodiments, the natural sweetener is luo han. In some embodiments, the concentration of the luo han in the gelatin capsule is about 0.00268%. In some embodiments, the dose comprises fish oil. In some embodiments, the dose comprises a sweetener, wherein the concentration by weight of the sweetener in the dose is from about 0.002% to about 0.003%. In some embodiments, the sweetener in the dose is a natural sweetener. In some embodiments, sweetener in the dose is luo han.

In some embodiments, the number of capsules in the plurality of capsules is about 30. In other embodiments, the number of capsules in the plurality of capsules is about 60. In yet other embodiments, number of capsules in the plurality of capsules is about 90. In yet other embodiments, number of capsules in the plurality of capsules is about 120. In yet other embodiments, number of capsules in the plurality of capsules is about 180. In yet other embodiments, the number of capsules in the plurality of capsules is from about 30 to about 180. In yet other embodiments, the number of capsules in the plurality of capsules is at least 30.

In another aspect, provided herein is a method of administering a nutritional or medicinal supplement that comprises administering a sweetened gelatin capsule to a subject. The subject may be in need of the supplement. In some embodiments, the gelatin capsule comprises a gelatin capsule encapsulating a dose, where the dose comprises a medicinal component or a nutritive component.

In another aspect, provided herein is a method of making a sweetened gelatin capsule that comprises encapsulating a dose within a sweetened gelatin capsule. In some embodiments, the dose comprises a fish oil. In some embodiments, the dose comprises a sweetener.

### DETAILED DESCRIPTION

Disclosed herein are embodiments of sweetened capsules for administration of a dose. In some embodiments, the gelatin capsule, the dose, or both the gelatin capsule and the dose comprise a sweetener. The dose may comprise a nutritive or medicinal component. In certain aspects, the sweetener can improve the palatability of the dose. In certain aspects, methods are disclosed for the administration of a sweetened capsule. In certain aspects, methods are disclosed for the making of a sweetened capsule. In certain aspects, a kit is disclosed for administration of a sweetened capsule.

### DEFINITIONS

About: In some embodiments, the term “about” as used herein, for example when referring to numerical attributes of gelatin capsules, means within ±5% of the nominal value. In other embodiments, the term “about” means within ±10% of the nominal value. In yet other embodiments, the term “about” means within ±20% of the nominal value.

### Capsules

In one aspect, capsules are disclosed for the administration of a dose. In an embodiment, capsule doses contain an active substance that is intended to be delivered into a digestive system. The dose may contain nutritive or medicinal components intended for human consumption. For example, the dose may be fish oil containing omega-3 fatty acids, which are known to be beneficial to human health. In another embodiment, the capsule may be intended for animal consumption.

Various capsule shapes are known in the art and are specifically contemplated. In one embodiment, a capsule can be spherically shaped. In another embodiment, a capsule can be ovally shaped. In yet another embodiment, a capsule can be cylindrically shaped with hemispherical ends. Those skilled in the art recognize that manufactured capsules are not exactly uniform in shape and can contain deviations from the ideal shape. Thus, capsules that are not perfectly spherically shaped, ovally shaped, or cylindrically shaped with hemispherical ends, are specifically contemplated.

It will also be recognized that a capsule need not be homogenous. For example, a capsule can comprise one, two, three, four, five, or more distinct components. In one embodiment, a capsule comprises at least one liquid component. In some embodiments, a capsule comprises two components, wherein a first component is encapsulated within a second component. Advantageously, the first component can be a liquid component, e.g., a fish oil, and the second component can be a solid component, e.g., a gelatin capsule.

Various sizes and weight of capsules are contemplated. In some embodiments, a capsule comprises about 500 milligrams (mg) of a dose and about 240 mg of an enclosing mechanism. In some embodiments, a capsule weighs from about 100 mg to 1 gram (g). In some embodiments, a capsule weighs from about 200 mg to 1800 mg. In some embodiments, a capsule weighs at least 100 mg, 200 mg, 250 mg, 300 mg, 400 mg, 500 mg, 750 mg, 1000 mg, 1250 mg, 1500 mg, or 1800 mg.

In certain aspects, the capsule comprises a solid component that encapsulates a dose and allows for oral consumption of the capsule. Once consumed, the solid component allows the dose to be released into the body. Such solid components can either be hard-shelled or soft-shelled. In a preferred embodiment, a sweetened gelatin capsule is used as the solid component. Preferably, the sweetener improves the taste of the capsule and/or the dose enclosed therein.

### Gelatin Capsules

In an embodiment, a sweetened gelatin capsule comprises an edible gelatin substance, water, a gelatin softener, and a sweetener. The edible gelatin substance can comprise gelatin or a gelatin substitute such as modified starch, vegetable gelatin, alginate, or other suitable edible gelatin substitute known in the art. In some embodiments, the concentration by weight of gelatin in a sweetened gelatin capsule is from about 10% to about 70%. In some embodiments, the concentration by weight of gelatin in a sweetened gelatin capsule is about 45%. In some embodiments, the sweetened gelatin capsules further comprises a coloring agent such as keratin, titanium oxide, or other suitable coloring agents known in the art.

Gelatin softeners are used in the art to soften the gelatin capsule and make it less brittle and more chewable. Gelatin softeners can include any glycerol, sorbitol, or other suitable polyol. In some embodiments, other gelatin softeners known in the art are used. In some embodiments, the concentration by weight of gelatin softener in a sweetened gelatin capsule is from about 10% to about 35%. In some embodiments, the concentration by weight of gelatin softener in a sweetened gelatin capsule ranging from about 10% to about 30%.

In some embodiments, the concentration by weight of water in a sweetened gelatin capsule is from about 10% to about 45%. In some embodiments, the concentration by weight of water in a sweetened gelatin capsule is from about 6% to about 10%. In some embodiments, the concentration by weight of water in a sweetened gelatin capsule is about 35%.

The sweetened gelatin capsules further comprise a sweetener. Sweeteners are sugar substitutes and can be derived from natural sources such as plants or artificially created. In some embodiments, a natural sweetener is used in the sweetened gelatin capsule. In other embodiments, an artificial sweetener like xylitol is used in the sweetened gelatin capsule. An example of a natural sweetener is luo han. Luo han sweetener is derived from the luo han guo fruit (siraitia grosvenorii) that is mainly found in China. It is about 300 times sweeter by weight than sucrose. Since luo han is much sweeter than sucrose, only a small amount is needed to make the gelatin capsule taste sweet. Luo han suitable for use as described herein is commercially available, for example, from Barrington Nutritionals (Harrison, N.Y.).

In some embodiments, luo han sweetener is used as the sweetener. In some embodiments, the concentration by weight of sweetener in a sweetened gelatin capsule is from about 0.002% to about 0.003%. In some embodiments, the concentration by weight of sweetener in a sweetened gelatin capsule is from about 0.0024% to about 0.0028%. In some embodiments, the concentration by weight of sweetener in a sweetened gelatin capsule about 0.00268%. In some embodiments, the concentration by weight of sweetener in a sweetened gelatin capsule is at least 0.001%, 0.002%, or 0.0025%. In some embodiments, there is about 10 mg of sweetener in a sweetened gelatin capsule encapsulating from about 400 milliliters (mL) to about 440 mL of a dose.

In some embodiments, the sweetened gelatin capsule further comprises a flavoring. The flavoring can further improve the taste of the capsule and the dose. An example of a flavoring agent is strawberry flavor Firmenich #52311A. Other suitable gelatin capsule flavorings known in the art can be used. In some embodiments, the flavoring is a fruit flavoring. In some embodiments, the flavoring comprises a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea. In some embodiments, the concentration by weight of flavoring in a sweetened gelatin capsule is from about 0.05% to about 5%. In some embodiments, the concentration by weight of flavoring in a sweetened gelatin capsule is from about 0.1% to about 4%, from about 0.1% to about 3%, from about 0.1% to about 2%, from about 0.1% to about 1.5%, from about 0.2% to about 4%, from about 0.2% to about 3%, from about 0.2% to about 2%, from about 0.2% to about 0.5%, from about 0.5% to about 5%, from about 0.5% to about 4%, from about 0.5% to about 3%, from about 0.5% to about 2%, from about 0.5% to about 1%, from about 1% to about 5%, from about 1% to about 4%, from about 1% to about 3%, or from about 1% to about 2%. In some embodiments, the concentration by weight of flavoring in a sweetened gelatin capsule is at least about 0.01%, about 0.1%, about 0.2%, about 0.3%, about 0.4%, or about 0.5%.

### The Dose

In another aspect, the gelatin capsule encloses a dose. The dose can comprise a nutritional or medicinal component, but is not limited to components of nutritional or medicinal value. For example, the dose could comprise a natural product, pharmaceutical, food supplement, diagnostic agent, vitamin, mineral, botanical product, herbal product, or marine product. In an embodiment, the dose comprises fish oil. Fish oil contains omega-3 fatty acids, which are polyunsaturated fatty acids, and have nutritional and medicinal value. In some embodiments, the fish oil comprises DHA, EPA, and other fatty acids. For example, a dose could comprise 330 mg of EPA and 220 mg of DHA. Persons of skill in the art would recognize that the relative concentrations of omega-3 compounds can be varied to maximize the benefit to different groups of people. For example, the relative concentrations of omega-3 compounds can differ for doses intended for women, pregnant women, children, or persons deficient in certain nutrients. In some embodiments, fish oil comprises about 80% of the total weight of the dose. In some embodiments, fish oil comprises from about 20% to about 90% of the total weight of the dose.

In some embodiments, a capsule comprises about 500 mg of a dose. In some embodiments, a capsule comprises from about 200 mg to about 800 mg of a dose. In some embodiments, a capsule comprises from about 50 mg to about 1.5 g of a dose. In some embodiments, a capsule comprises at least 50 mg, 100 mg, 200 mg, 250 mg, 300 mg, 400 mg, 500 mg, 750 mg, 1000 mg, or 1200 mg of a dose.

In some embodiments, the dose further comprises a sweetener. In some embodiments, the sweetener in the dose is a natural sweetener. In some embodiments, the sweetener in the dose is luo han. In some embodiments, the concentration by weight of sweetener in a dose is from about 0.002% to about 0.003%. In some embodiments, the concentration by weight of sweetener in dose is from about 0.0024% to about 0.0028%. In some embodiments, the concentration by weight of sweetener in a dose is about 0.00268%. In some embodiments, the concentration by weight of sweetener in a dose is at least 0.001%, 0.002%, or 0.0025%.

In certain embodiments, both the dose and the gelatin capsule encapsulating the dose comprise a sweetener. In some of these embodiments, the type of sweetener in the gelatin capsule is the same type of sweetener in the dose. In other embodiments, the type of sweetener in the gelatin capsule is different from the type of sweetener in the dose. In some embodiments, the concentration by weight of sweetener in the dose is about equal to the concentration by weight of sweetener in the gelatin capsule.

In some embodiments, the dose further comprises a flavoring. The flavoring can further improve the taste of the dose. In some embodiments, the flavoring in the dose is a fruit flavoring. In some embodiments, the flavoring comprises a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea. In some embodiments, the concentration by weight of flavoring in a dose is from about 0.05% to about 5%. In some embodiments, the concentration by weight of flavoring in a dose is from about 0.05% to about 5%. In some embodiments, the concentration by weight of flavoring in a sweetened gelatin capsule is from about 0.1% to about 4%, from about 0.1% to about 3%, from about 0.1% to about 2%, from about 0.1% to about 1.5%, from about 0.2% to about 4%, from about 0.2% to about 3%, from about 0.2% to about 2%, from about 0.2% to about 0.5%, from about 0.5% to about 5%, from about 0.5% to about 4%, from about 0.5% to about 3%, from about 0.5% to about 2%, from about 0.5% to about 1%, from about 1% to about 5%, from about 1% to about 4%, from about 1% to about 3%, or from about 1% to about 2%. In some embodiments, the concentration by weight of flavoring in a dose is at least about 0.01%, about 0.1%, about 0.2%, about 0.3%, about 0.4%, or about 0.5%.

In some embodiments, both the dose and the gelatin capsule encapsulating the dose comprise a flavoring. In some of these embodiments, the flavoring in the gelatin capsule is the same flavoring in the dose. In other embodiments, the flavoring in the gelatin capsule is different from the flavoring in the dose. In some embodiments, the concentration by weight of the flavoring in the dose is about equal to the concentration by weight of flavoring in the gelatin capsule. In other embodiments where the dose comprises a flavoring, the gelatin capsule enclosing the dose does not comprise a flavoring.

### Methods Relating to Sweetened Gelatin Capsules

In one aspect, provided herein is a method making a sweetened gelatin capsule. One method comprises encapsulating a dose within a sweetened gelatin capsule. A gelswatch comprises a mixture of a gelatin substance, gelatin softener, water, and sweetener. Optionally, the gelswatch can also contain a flavoring or a coloring agent. The gelswatch can be heated to about 60 degrees to 70 degrees Celsius and mixed together. The gelatin capsule will be made from the gelswatch material. The liquefied gelswatch and the dose can then be poured into an encapsulation machine. The encapsulation machine forms the sweetened gelatin capsule comprising the dose encapsulated by the gelatin capsule. The capsule can then be dried at a temperature of, for example, about 20 degrees Celsius. The water content of the capsule may be reduced by evaporation during the drying step. The capsule can then be washed and ready for packaging, selling, or shipping. In some embodiments, the gelswatch does not comprise sweetener and the sweetener is added to the capsule through a dipping process. In the dipping process, the gelatin capsule is dipped in a sweetener solution (that my also optionally comprise a flavor) and then dried, allowing for the sweetener to form a coating around the outside of the capsule. In some embodiments, the gelswatch does not comprise sweetener and the sweetener is added to the capsule through an enteric coating process. In other embodiments, a liquefied sweetener can be sprayed on to the outside of the gelatin capsule and dried. Other methods of making gelatin capsules are known in the art and contemplated.

In another aspect, provided herein is a method of administering a nutritional or medicinal supplement. In one embodiment, the method comprises administering a sweetened gelatin capsule to a subject, wherein the dose encapsulated in the capsule contains a nutritional or medicinal component.

### Kits

In yet another aspect, provided herein is a kit for providing a nutritional or medicinal supplement. In certain embodiments, the kit comprises a plurality of capsules and a container for storing the plurality of capsules. The capsules comprise a dose encapsulated in a gelatin capsule. Embodiments of capsules for use in a kit include the embodiments disclosed herein. For example, each capsule in the plurality of capsules can comprise fish oil encapsulated in a sweetened gelatin capsule. An example of a container is a plastic screw-top container. Persons of skill in the art recognize that the container can come in different shapes, colors, and materials.

In some embodiments, the kit comprises about 60 capsules in a container. In some embodiments, the kit comprises about 90 capsules in a container. In some embodiments, the kit comprises about 120 capsules in a container. In some embodiments, the kit comprises at least 10 capsules, 20 capsules, 30 capsules, 40 capsules, 50 capsules, or 60 capsules in a container. In some embodiments, the kit comprises from about 50 capsules to about 150 capsules in a container.

### EXAMPLE OF SWEETENER CONCENTRATIONS

A taste test was conducted with different concentrations of sweetener mixed into cod liver oil (CLO). The sweetener used was luo han, and the CLO had a 2% concentration by weight of strawberry flavoring mixed into it. There were five samples used in the taste test. The amount of luo han was kept constant at 10 mg, while the amount of CLO varied from 400 mL to 440 mL in increments of 10 mL. Thus the concentration by weight of luo han varied from 0.00268% (in 400 mL of CLO) to 0.00243% (in 440 mL of CLO). The subjects of the taste test were children and adults. The children were comprised of two children of each age listed: 5, 6, 7, 8, 9, and 10. The number of adults totaled eight. The taste test subjects preferred the highest concentration of luo han that was available in the test, i.e. 10 mg of luo han in 400 mL of strawberry-flavored CLO.

Many modifications and variations of this application can be made without departing from its spirit and scope, as will be apparent to those skilled in the art. The specific embodiments described herein are offered by way of example only, and the application is to be limited only by the terms of the appended claims, along with the full scope of equivalents to which such claims are entitled.

What is claimed:

1. A sweetened gelatin capsule suitable for encapsulating a dose, the sweetened gelatin capsule comprising:
a. a gelatin substance;
b. a gelatin softener;
c. water, wherein the concentration by weight of the water in the sweetened gelatin capsule is between 6% and 10%; and
d. a sweetener, wherein the concentration by weight of the sweetener in the sweetened gelatin capsule is from about 0.002% to about 0.003%.

2. The sweetened gelatin capsule of claim 1, wherein the sweetener is a natural sweetener.

3. The sweetened gelatin capsule of claim 2, wherein the natural sweetener is luo han.

4. The sweetened gelatin capsule of claim 3, wherein the concentration by weight of the luo han in the sweetened gelatin capsule is about 0.00268%.

5. The sweetened gelatin capsule of claim 1, wherein the concentration by weight of the sweetener in the sweetened gelatin capsule is from about 0.0024% to about 0.0028%.

6. The sweetened gelatin capsule of claim 1, wherein the sweetened gelatin capsule encapsulates a dose.

7. The sweetened gelatin capsule of claim 6, wherein the dose comprises fish oil.

8. The sweetened gelatin capsule of claim 6, wherein the dose comprises a sweetener, wherein the concentration by weight of the sweetener in the dose is between 0.002% and 0.003%.

9. The sweetened gelatin capsule of claim 8, wherein the sweetener in the dose is a natural sweetener.

10. The sweetened gelatin capsule of claim 9, wherein the natural sweetener in the dose is luo han.

11. The sweetened gelatin capsule of claim 6, wherein the dose comprises a flavoring.

12. The sweetened gelatin capsule of claim 11, wherein the concentration by weight of the flavoring in the dose is from about 0.05% to about 5%.

13. The sweetened gelatin capsule of claim 11, wherein the flavoring comprises a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea.

14. The sweetened gelatin capsule of claim 11, wherein the flavoring is a fruit flavoring.

15. The sweetened gelatin capsule of claim 6, wherein the dose comprises a medicinal component or a nutritive component.

16. The sweetened gelatin capsule of claim 1, wherein the sweetened gelatin capsule further comprises a flavoring.

17. The sweetened gelatin capsule of claim 16, wherein the concentration by weight of the flavoring in the sweetened gelatin capsule is from about 0.05% to about 5%.

18. The sweetened gelatin capsule of claim 16, wherein the flavoring comprises a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea.

19. The sweetened gelatin capsule of claim 16, wherein the flavoring is a fruit flavoring.

20. The sweetened gelatin capsule of claim 1, wherein the concentration by weight of the gelatin softener in the sweetened gelatin capsule is from about 10% to about 35%.

21. The sweetened gelatin capsule of claim 1, wherein the sweetened gelatin capsule further comprises a coloring agent.

22. A sweetened gelatin capsule, the sweetened gelatin capsule comprising:
a. a dose; and
b. a gelatin capsule encapsulating the dose, the gelatin capsule comprising:
i. a gelatin substance;
ii. a gelatin softener;
iii. water, wherein the concentration by weight of the water in the gelatin capsule is from about 6% to about 10%; and
iv. a sweetener, wherein the concentration by weight of the sweetener in the gelatin capsule is from about 0.002% to about 0.003%.

23. The sweetened gelatin capsule of claim 22, wherein the sweetener is a natural sweetener.

24. The sweetened gelatin capsule of claim 23, wherein the natural sweetener is luo han.

25. The sweetened gelatin capsule of claim 24, wherein the concentration by weight of the luo han in the gelatin capsule is about 0.00268%.

26. The sweetened gelatin capsule of claim 22, wherein the concentration by weight of the sweetener in the gelatin capsule is from about 0.0024% to about 0.0028%.

27. The sweetened gelatin capsule of claim 22, wherein the dose comprises fish oil.

28. The sweetened gelatin capsule of claim 22, wherein the dose comprises a sweetener.

29. The sweetened gelatin capsule of claim 28, wherein the concentration by weight of the sweetener in the dose is between 0.002% and 0.003%.

30. The sweetened gelatin capsule of claim 28, wherein the concentration by weight of the sweetener in the dose is about equal to the concentration by weight of the sweetener in the gelatin capsule.

31. The sweetened gelatin capsule of claim 28, wherein the sweetener in the dose is a natural sweetener.

32. The sweetened gelatin capsule of claim 31, wherein the natural sweetener in the dose is luo han.

33. The sweetened gelatin capsule of claim 22, wherein the dose comprises a flavoring.

34. The sweetened gelatin capsule of claim 33, wherein the concentration by weight of the flavoring in the dose is from about 0.05% to about 5%.

35. The sweetened gelatin capsule of claim 33, wherein the flavoring comprises a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea.

36. The sweetened gelatin capsule of claim 33, wherein the flavoring is a fruit flavoring.

37. The sweetened gelatin capsule of claim 22, wherein the gelatin capsule further comprises a flavoring.

38. The sweetened gelatin capsule of claim 37, wherein the concentration by weight of the flavoring in the gelatin capsule is from about 0.05% to about 5%.

39. The sweetened gelatin capsule of claim 37, wherein the flavoring comprises a flavor selected from the group consisting of berry, strawberry, chocolate, cocoa, lemon, almond, cashew, macadamia nut, coconut, blueberry, blackberry, raspberry, peach, lemon, lime, mint, orange, banana, chili pepper, pepper, cinnamon, pineapple, vanilla, vanillin, apple, tooty fruity, mango, and green tea.

40. The sweetened gelatin capsule of claim 37, wherein the flavoring is a fruit flavoring.

41. The sweetened gelatin capsule of claim 22, wherein the concentration by weight of the gelatin softener in the gelatin capsule is from about 10% to about 35%.

42. The sweetened gelatin capsule of claim 22, wherein the dose comprises a medicinal component or a nutritive component.

43. The sweetened gelatin capsule of claim 22, wherein the gelatin capsule further comprises a coloring agent.

44. A kit for providing a nutritional or medicinal supplement, the kit comprising:
a. a plurality of capsules, wherein each capsule in the plurality of capsules comprises a gelatin capsule encapsulating a dose, the gelatin capsule comprising:
i. a gelatin substance;
ii. a gelatin softener;
iii. water, wherein the concentration by weight of the water in the gelatin capsule is from about 6% to about 10%; and
iv. a sweetener, wherein the concentration by weight of the sweetener in the gelatin capsule is from about 0.002% to about 0.003%; and
b. a container for storing the plurality of capsules.

45. The kit of claim 44, wherein the sweetener is a natural sweetener.

46. The kit of claim 45, wherein the natural sweetener is luo han.

47. The kit of claim 46, wherein the concentration of the luo han in the gelatin capsule is about 0.00268%.

48. The kit of claim 44, wherein the dose comprises fish oil.

49. The kit of claim 44, wherein the dose comprises a sweetener, wherein the concentration by weight of the sweetener in the dose is from about 0.002% to about 0.003%.

50. The kit of claim 49, wherein the sweetener in the dose is a natural sweetener.

51. The kit of claim 50, wherein the sweetener in the dose is luo han.

52. The kit of claim 44, wherein the number of capsules in the plurality of capsules is about 60.

53. The kit of claim 44, wherein the number of capsules in the plurality of capsules is about 90.

54. The kit of claim 44, wherein the number of capsules in the plurality of capsules is about 120.

55. The kit of claim 44, wherein the number of capsules in the plurality of capsules is from about 50 to about 150.

56. The kit of claim 44, wherein the number of capsules in the plurality of capsules is at least 30.

57. The kit of claim 44, wherein the concentration by weight of the gelatin softener in the gelatin capsule is from about 10% to about 35%.

58. A method of administering a nutritional or medicinal supplement, the method comprising administering the sweetened gelatin capsule of claim 22 to a subject.

59. The method of claim 58, wherein the dose of claim 22 comprises a medicinal component or a nutritive component.

60. A method of making a sweetened gelatin capsule, the method comprising encapsulating a dose within the sweetened gelatin capsule of claim 1.

61. The method of claim 60, wherein the dose comprises a fish oil.

62. The method of claim 60, wherein the dose comprises a sweetener.
